(19) United States
(12) Patent Application Publication
(10) Pub. No.: US 2013/0261349 A1
(43) Pub. Date: Oct. 3, 2013

(54) Hydrogenation Catalysts and Processes for Making Same

(71) Applicants: Heiko Weiner, Pasadena, TX (US); Zhenhua Zhou, Houston, TX (US)

(72) Inventors: Heiko Weiner, Pasadena, TX (US); Zhenhua Zhou, Houston, TX (US)

(73) Assignee: CELANESE INTERNATIONAL CORPORATION, Dallas, TX (US)

(21) Appl. No.: 13/432,884

(22) Filed: Mar. 28, 2012

(51) Int. Cl. C07C 27/04; B01J 21/08; B01J 23/652; B01J 23/89; B01J 23/14; B01J 23/835; B01J 23/648; B01J 23/26; B01J 23/28; B01J 23/30; B01J 23/22; B01J 37/02; B01J 37/08; B01J 37/04; B01J 23/62

(52) U.S. Cl. 568/885; 502/339; 502/242; 502/310; 502/326; 502/331; 502/329; 502/352; 502/325; 502/345; 502/343

(57) ABSTRACT

A catalyst composition comprising tin and optionally a second metal for use in the production of alcohols such as ethanol from carboxylic acids such as acetic acid. An acidic solution such as nitric acid is utilized in the preparation of the catalyst according to one embodiment of the present invention to better solubilize an organometallic tin precursor resulting in the formation of catalysts having particularly high selectivity to ethanol.

### FIELD OF THE INVENTION

The present invention relates generally to catalysts, to processes for producing catalysts, and to processes for using such catalysts. More specifically, the present invention relates to tin-containing catalysts and to processes for making and using tin-containing catalysts.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane via fermentation routes. Conventional methods for producing ethanol from petrochemical feed stocks, as well as from cellulose materials, include the acid-catalyzed hydration of ethylene, methanol homologation, and direct alcohol synthesis. Instability in petrochemical feed stock prices contributes to fluctuations in the cost of conventionally produced ethanol, rendering the need for alternative sources of ethanol production greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed, for example, by EP0175558 and U.S. Pat. No. 4,398,039. A summary of the developmental efforts for hydrogenation catalysts for conversion of various carboxylic acids is provided in Yokoyama, et al., “Carboxylic acids and derivatives,” Fine Chemicals Through Heterogeneous Catalysis, 2001, 370-379.

More recently, it has been reported that ethanol can be produced from hydrogenating acetic acid using a cobalt catalyst at superatmospheric pressures such as about 40 to 120 bar. See, for example, U.S. Pat. No. 4,517,391 to Shuster et al. However, the only example disclosed therein employs reaction pressure in the range of about 300 bar still making this process undesirable for commercial operation. In addition, the process calls for a catalyst containing no less than 50 percent cobalt by weight plus one or more members selected from the group consisting of copper, manganese, molybdenum, chromium, and phosphoric acid, thus rendering the process economically non-viable.

U.S. Pat. No. 5,149,680 to Kitson et al. describes a process for the catalytic hydrogenation of carboxylic acids and their anhydrides to alcohols and/or esters utilizing metal alloy catalysts. The catalyst is comprised of an alloy of at least one noble metal of Group VIII of the Periodic Table and at least one metal capable of alloying with the Group VIII noble metal, admixed with a component comprising at least one of the metals rhenium, tungsten or molybdenum. Although it has been claimed therein that improved selectivity to alcohols are achieved over prior art references, alkane production was still reported. In particular, methane and ethane were formed as by-products during the hydrogenation of acetic acid to ethanol under their optimal catalyst conditions.

U.S. Pat. No. 4,777,303 to Kitson et al. describes a process for the production of alcohols by the hydrogenation of carboxylic acids. The catalyst used in the '303 patent was a heterogeneous catalyst comprising a first component which is either molybdenum or tungsten and a second component which is a noble metal of Group VIII of the Periodic Table of the Elements, optionally on a support, for example, on high surface area graphitized carbon. The selectivity to a combined mixture of alcohol and ester is reported to be in the range of about 73 to 87 percent with low conversion of carboxylic acids at about 16 to 58 percent. In addition, no specific example of conversion of acetic acid to ethanol is provided.

U.S. Pat. No. 4,804,791 to Kitson et al. describes another process for the production of alcohols by the hydrogenation of carboxylic acids. In this process, ethanol is produced from acetic acid or propanol is produced from propionic acid by contacting either acetic acid or propionic acid in the vapor phase with hydrogen at elevated temperature and a pressure in the range from 1 to 150 bar in the presence of a catalyst comprising as essential components (i) a noble metal of Group VIII of the Periodic Table of the elements, and (ii) rhenium, optionally on a support, for example a high surface area graphitized carbon. The conversion of acetic acid to ethanol ranged from 0.6% to 69% with selectivity to ethanol was in the range of about 6% to 97%.

Malinowski et al., Bull. Soc. Chim. Belg. (1985), 94(2), 93-5, the entirety of which is incorporated herein by reference, discuss reaction catalysis of acetic acid on low-valence titanium heterogenized on support materials such as silica (SiO₂) or titania (TiO₂).

Bimetallic ruthenium-tin/silica catalysts have been prepared by reaction of tetrabutyl tin with ruthenium dioxide supported on silica. See Loessard et al., “Studies in Surface Science and Catalysis,” 48 Struct. React. Surf at 591-600 (1989). Additional hydrogenation catalysts are further described in U.S. Pat. No. 7,608,744, U.S. Pub. No. 2009/0069609 and U.S. Pub. No. 2010/0029995, the entireties of which are incorporated herein by reference.

The catalytic reduction of acetic acid has also been studied by, for instance, Hindermann et al., who disclose the catalytic reduction of acetic acid on iron and on alkali-promoted iron. See Hindermann et al., J. Chem. Res., Synopses (11), 373 (1980), the entirety of which is incorporated herein by reference.

In view of the foregoing, the need exists for novel hydrogenation catalysts that have high selectivity, conversion, and productivity to ethanol and having catalyst lifetimes that are suitable for commercial hydrogenation processes. The need also exists for methods for preparing such catalysts.

### SUMMARY OF THE INVENTION

The present invention relates to processes for producing catalysts that are preferably suitable for catalyzing the hydrogenation of one or more carboxylic acids, such as acetic acid, to one or more alcohols, such as ethanol. The catalysts preferably are particularly robust and enable high conversions and selectivity to ethanol.

In one embodiment, the process comprises the steps of (a) contacting a catalyst support with a precursor solution comprising a tin precursor, a Group VIII metal precursor, and water at a pH less than 3, e.g., less than 2.5 or less than 2, to form an impregnated support, and (b) drying the impregnated support to form a catalyst comprising tin and a Group VIII metal on the support. The precursor solution preferably is substantially free of ammonium oxalate. The precursor solution may comprise nitric acid. The catalyst may have a productivity for ethanol of at least 700 grams of ethanol per kg catalyst per hour. The contacting step may be performed by spraying the catalyst support with the precursor solution, by incipient wetness techniques, or by adding the catalyst support to the precursor solution. The catalyst support may comprise a mixture of silica and calcium silicate. The catalyst may comprise from 0.1 to 10 wt. % platinum or palladium and from 0.1 to 10 wt. % tin. The catalyst may be calcined at a temperature between 250° C. and 650° C.

In another embodiment, the invention is to a process for preparing a catalyst, the process comprising the steps of: (a) providing a first solution comprising a tin precursor and an acid selected from nitric acid, hydrochloric acid, sulfuric acid, acetic acid, citric acid, formic acid, lactic acid, malic acid, orthophosphoric acid, salicylic acid, succinic acid, tartaric acid and trichloroacetic acid; (b) combining a second metal precursor or a solution thereof with the first solution to form a mixed metal precursor solution, wherein the second metal precursor comprises a second metal oxalate, acetate, halide or nitrate; (c) impregnating a support with the mixed metal precursor solution to form an impregnated support; and (d) drying the impregnated support to form a catalyst comprising tin and the second metal on the support. The first solution, and mixed metal precursor preferably has a pH less than 3, e.g., less than 2.5 or less than 2. The precursor solution may comprise nitric acid. The catalyst may have a productivity for ethanol of at least 700 grams of ethanol per kg catalyst per hour. The contacting step may be performed by spraying the catalyst support with the precursor solution, by incipient wetness techniques, or by adding the catalyst support to the precursor solution. The catalyst support may comprise a mixture of silica and calcium silicate. The catalyst may comprise from 0.1 to 10 wt. % platinum or palladium and from 0.1 to 10 wt. % tin. The catalyst may be calcined at a temperature between 250° C. and 650° C.

In one aspect, the invention is to a process for producing ethanol comprising the steps of: (a) contacting a catalyst support with a catalyst precursor mixture comprising a tin precursor, a second metal precursor and an acidic impregnation solution such that pores of the catalyst support are filled with the catalyst precursor mixture; and (b) drying the catalyst precursor mixture to form a catalyst composition comprising tin and the second metal; and (c) hydrogenating a feed stream comprising acetic acid and hydrogen over the catalyst composition to produce a crude ethanol product stream, wherein the impregnation solution comprises nitric acid. The selectivity to ethanol based on acetic acid consumed may be at least 60%, e.g., at least 80%. The catalyst precursor mixture preferably has a pH less than 3, e.g., less than 2.5 or less than 2.

In another embodiment, the invention is to a process for producing ethanol comprising the steps of: (a) providing a catalyst composition comprising tin and a second metal, the catalyst composition being formed by contacting a catalyst support with a catalyst precursor mixture comprising a tin precursor and a second metal precursor in an acidic impregnation solution such that pores of the catalyst support are filled with the catalyst precursor mixture, and drying the catalyst precursor mixture to form the catalyst composition; and (b) hydrogenating a feed stream comprising acetic acid and hydrogen over the catalyst composition to produce a crude ethanol product stream. The catalyst precursor mixture may comprise nitric acid and preferably has a pH less than 3, e.g., less than 2.5 or less than 2.

### DETAILED DESCRIPTION OF THE INVENTION

### Introduction

The present invention relates to catalysts that are preferably suitable for catalyzing the hydrogenation of a carboxylic acid, such as acetic acid, to its corresponding alcohol, such as ethanol. The catalyst comprises tin and preferably a second metal, which optionally is selected from any Group IB, IIB, IIIB, IVB, VB, VIIB, VIIB, VIII transition metal, a lanthanide metal, an actinide metal or a metal selected from any of Groups IIIA, IVA (other than tin), VA, and VIA. The catalyst is preferably a supported catalyst in which tin is dispersed on the catalyst support. The catalyst may further comprise a support modifier.

The present invention also relates to processes for making the catalysts of the invention utilizing a low pH catalyst preparation solution, preferably an aqueous nitric acid solution, having a pH of less than 3, e.g., less than 2.5 or less than 2.

In another embodiment, the present invention further relates to processes for producing ethanol by hydrogenating acetic acid in the presence of the inventive catalyst. It has surprisingly and unexpectedly been discovered that the catalysts of the present invention provide high selectivities to ethanol when employed, for example, in the hydrogenation of acetic acid to form ethanol. Embodiments of the present invention beneficially may be used in industrial applications to produce ethanol on an economically feasible scale. For example, depending on the temperature at which the hydrogenation reaction is conducted, high alkanoic acid, e.g., acetic acid, conversions of at least 50 mol %, at least 80 mol %, at least 90 mol %, or at least 95 mo % may be achieved with the inventive catalyst compositions. These high conversions are desirably achieved while maintaining high selectivity to the desired alcohol, e.g., ethanol.

### Catalyst Compositions

Catalytically active materials act through their surfaces. As a result, the surface of the catalyst must be accessible to the reactants in order to ensure high catalytic activity. Without being bound by theory, such accessibility can be advantageously maximized by dispersing the active components as particles (sometimes as small as a few nanometers), and stabilizing them on the surface of a catalyst support material.

Suitable hydrogenation catalysts include catalysts comprising tin and optionally one or more of a second metal, a third metal or any number of additional metals, optionally on a catalyst support. The optional second, third and additional metals may be selected from Group IB, IIB, IIIB, IVB, VB, VIIB, VIIB, VIII transition metals, a lanthanide metal, an actinide metal or a metal selected from any of Groups IIIA, IVA, VA, and VIA (other than tin). In one embodiment, the second metal is a Group VIII metal, optionally selected from platinum and palladium. In another embodiment, the catalyst comprises tin and a second metal selected from cobalt, platinum, palladium, nickel, ruthenium, copper, iron, cobalt, rhodium, osmium, iridium, titanium, zinc, chromium, rhenium, molybdenum, and tungsten. In embodiments of the invention where the second metal comprises platinum, rhodium, or gold it is preferred that the catalyst comprises the platinum, rhodium or gold in an amount less than 5 wt. %, e.g., less than 3 wt. % or less than 1 wt. %, due to the high commercial prices for these metals. The catalyst may also comprise a third metal selected from any of the metals listed above in connection with the second metal, so long as the third metal is not tin and is different from the second metal. Thus, in one embodiment, the catalyst comprises tin, a second metal as described above, and a third metal selected from cobalt, platinum, palladium, nickel, ruthenium, copper, iron, cobalt, rhodium, osmium, iridium, titanium, zinc, chromium, rhenium, molybdenum, and tungsten, so long as the third metal is different from the second metal. Preferred metal combinations for some exemplary catalyst compositions include cobalt/tin, platinum/tin, palladium/tin, tin/platinum/ruthenium, tin/platinum/rhenium, tin/palladium/ruthenium, tin/palladium/rhenium, tin/cobalt/palladium, tin/cobalt/platinum, tin/cobalt/chromium, tin/cobalt/ruthenium, cobalt/tin, tin/silver/palladium, tin/copper/palladium, tin/copper/zinc, tin/nickel/palladium, tin/gold/palladium, tin/ruthenium/rhenium, and tin/ruthenium/iron.

In certain embodiments where the catalyst includes two or more metals, e.g., tin and a second metal, the second metal may be present in the catalyst in an amount from 0.1 to 10 wt. %, e.g., from 0.1 to 5 wt. %, or from 0.1 to 3 wt. %. The tin may be present in an amount greater than 0.01 wt. %, greater than 0.1 wt. % or greater than 1 wt. %. In terms of ranges, the tin may be present in an amount from 0.1 to 20 wt. %, e.g., from 0.1 to 10 wt. %, or from 0.1 to 8 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the second metal to the tin is from 10:1 to 1:20, e.g., from 4:1 to 1:4, from 2:1 to 1:2, from 1.5:1 to 1:1.5 or from 1.1:1 to 1:1.1. In one embodiment, the tin is present in a greater molar amount than the second metal, for example, at a second metal to tin mole ratio of from 1:1 to 1:25, e.g., from 1:8 to 1:20 or from 1:10 to 1:15.

In preferred aspects, if present, the third metal is selected from the group consisting of cobalt, palladium, ruthenium, copper, zinc, platinum, and rhenium. More preferably, the third metal is selected from cobalt, palladium, and ruthenium. When present, the total weight of the third metal preferably is from 0.05 to 10 wt. %, e.g., from 0.1 to 9, or from 5 to 8.

The metals in the catalyst may be present in the form of one or more metal oxides. For purposes of determining the weight percent of the metals in the catalyst, the weight of any oxygen that is bound to the metal is ignored.

Without being bound by theory, one metal may act as a promoter metal and the other metal as the main metal. For instance, with a tin and platinum or palladium catalyst, tin may be considered as the main metal and platinum or palladium may be considered as the promoter metal. For convenience, the present specification refers to tin as the main metal and platinum and palladium as promoters. This should not be taken as an indication of the underlying mechanism of the catalytic activity.

Other exemplary promoters that may be included in the inventive catalyst include lithium, sodium, magnesium, aluminum, manganese, iron, cobalt, calcium, yttrium, silver, barium, lanthanum, the rare earth metals, hafnium, tantalum, rhenium, thorium, bismuth, antimony, germanium, zirconium, uranium, cesium, and silicon and combinations thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

Depending primarily on how the catalyst is manufactured, the metal(s) of the catalysts of the present invention may be dispersed throughout the support, coated on the outer surface of the support (i.e., egg shell) or decorated on the surface of the support. According to preferred embodiments of the present invention, however, the tin is dispersed on the catalyst support.

In addition to tin and optionally one or more additional metals, in some embodiments of the present invention the catalysts further comprise a support or a modified support. As used herein, the term “modified support” refers to a support that includes a support material and a support modifier, which adjusts the acidity of the support material.

As will be appreciated by those of ordinary skill in the art, support materials are selected such that the catalyst system is suitably active, selective and robust under the process conditions employed for the formation of ethanol. The overall success of a catalyst depends on a number of factors, including: (1) the ability of the catalyst support to provide a large enough surface area to stabilize the catalyst (e.g., against sintering); (2) the extent of the catalyst support's porous structure, which generally should allow for easy access for process reactants and products to diffuse in and out; (3) the nature and strength of interactions between the catalyst support and the catalytically active phase; (4) the ability to form the catalyst support in various sizes or shapes required for optimal mass and heat transfer during catalyst operations under reactor conditions, as the case may be; and (5) other factors, such as mechanical strength, chemical and structural stability, thermal stability, resistance to attrition, and ease of availability at a high volume and/or low cost, particularly in commercial settings.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

In the case where silica is used as the silicaceous support, it is beneficial to ensure that the amount of aluminum, which is a common contaminant for silica, is low, preferably under 1 wt. %, e.g., under 0.5 wt. % or under 0.3 wt. %, based on the total weight of the modified support. In this regard, pyrogenic silica is preferred as it commonly is available in purities exceeding 99.7 wt. %. High purity silica, as used throughout the application, refers to silica in which acidic contaminants such as aluminum are present, if at all, at levels of less than 0.3 wt. %, e.g., less than 0.2 wt. % or less than 0.1 wt. %.

In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

The acid sites, e.g., Brønsted acid sites, on the support material may be adjusted by the support modifier to favor selectivity to ethanol during the hydrogenation of acetic acid. The acidity of the support material may be adjusted by reducing the number or reducing the availability of Brønsted acid sites on the support material. The support material may also be adjusted by having the support modifier change the pKa of the support material. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference. It has now been discovered that in addition to the metal precursors and preparation conditions employed, metal-support interactions may have a strong impact on selectivity to ethanol. In particular, the use of modified supports that adjust the acidity of the support to make the support less acidic or more basic surprisingly and unexpectedly has now been demonstrated to favor formation of ethanol over other hydrogenation products.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier may be present in an amount from 0.1 to 50 wt. %, e.g., from 0.2 to 25 wt. %, from 0.5 to 15 wt. %, or from 1 to 8 wt. %, based on the total weight of the catalyst. In preferred embodiments, the support material is present in an amount from 25 wt. % to 99 wt. %, e.g., from 30 wt. % to 97 wt. % or from 35 wt. % to 95 wt. %, based on the total weight of the catalyst.

A preferred silica support material is SS61138 High Surface Area (HSA) Silica Catalyst Carrier from Saint Gobain N or Pro. The Saint-Gobain N or Pro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The surface area of the support material, e.g., silicaceous support material, preferably is at least 50 m²/g, e.g., at least 100 m²/g, at least 150 m²/g, at least 200 m²/g or most preferably at least 250 m²/g. In terms of ranges, the silicaceous support material, e.g., silica, preferably has a surface area of from 50 to 600 m²/g, e.g., from 100 to 500 m²/g or from 100 to 300 m²/g. High surface area silica, as used throughout the application, refers to silica having a surface area of at least 250 m²/g. For purposes of the present specification, surface area refers to BET nitrogen surface area, meaning the surface area as determined by ASTM D6556-04, the entirety of which is incorporated herein by reference.

The support material, e.g., silica, also preferably has an average pore diameter of from 5 to 100 nm, e.g., from 5 to 30 nm, from 5 to 25 nm or from about 5 to 10 nm, as determined by mercury intrusion porosimetry, and an average pore volume of from 0.5 to 2.0 cm³/g, e.g., from 0.7 to 1.5 cm³/g or from about 0.8 to 1.3 cm³/g, as determined by mercury intrusion porosimetry.

The morphology of the support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silicaceous support material has a morphology that allows for a packing density of from 0.1 to 1.0 g/cm³, e.g., from 0.2 to 0.9 g/cm³ or from 0.5 to 0.8 g/cm³. In terms of size, the silica support material preferably has an average particle size, e.g., meaning the diameter for spherical particles or average longest dimension for non-spherical particles, from 0.01 to 1.0 cm, e.g., from 0.1 to 0.5 cm or from 0.2 to 0.4 cm. Since the one or more metal(s) that are disposed on or within the modified support are generally very small in size, they should not substantially impact the size of the overall catalyst particles. Thus, the above particle sizes generally apply to both the size of the modified supports as well as to the final catalyst particles.

The use and presence of a support in the context of supported catalysts offer several advantages. First, supports can provide the catalyst with various mechanical properties needed for a long-lasting operation (e.g., hardness, resistance to crushing or erosion). Second, catalyst supports also improve the high dispersion of the active phase. Furthermore, supported catalysts may also demonstrate increased stability against sintering due to separation and dilution of metal particles on the support, and to metal-support interactions. In view of the above, the use of supports can contribute to a better utilization of the catalytically active phase by maximizing the active phase surface area exposed to the reactants. Support materials also tend to be less expensive than most catalytic metals, thereby helping to keep capital expenditures low. Additional improvements with respect to selectivity, activity and catalyst regenerability may also be achieved by a careful selection of the support.

In some embodiments where it is desired for the catalyst to produce ethanol at high selectivity, as indicated above, controlling the Brønsted acidity of the support material by incorporating a support modifier can be quite beneficial. One possible byproduct of the hydrogenation of acetic acid is ethyl acetate. In some embodiments, the support includes a basic support modifier that is effective to suppress production of ethyl acetate, rendering the catalyst composition highly selective to ethanol. Thus, the catalyst composition preferably has a low selectivity to ethyl acetate and highly undesirable by-products such as alkanes. The acidity of the support preferably is controlled such that less than 4%, preferably less than 2% and most preferably less than about 1% of the acetic acid is converted to methane, ethane and carbon dioxide. In addition, the acidity of the support may be controlled by using a pyrogenic silica or high purity silica as discussed above.

Basic support modifiers such as calcium metasilicate may tend to have a lower surface area than the support material, e.g., silicaceous support material, reducing overall modified support surface area. In one embodiment, the support material comprises a silicaceous support material that includes at least about 80 wt. %, e.g., at least about 85 wt. % or at least about 90 wt. %, high surface area silica in order to counteract this effect of including a support modifier. In one embodiment, the calcium metasilicate is present in an amount from 1 wt. % to 10 wt. %, based on the total weight of the catalyst.

Accordingly, without being bound by theory, modification and stabilization of oxidic support materials for the catalysts of the present invention by incorporation of non-volatile support modifiers having either the effect of: counteracting acid sites present upon the support surface or the effect of thermally stabilizing the surface makes it possible to achieve desirable improvements in selectivity to ethanol, prolonged catalyst life, or both. In general, support modifiers based on oxides in their most stable valence state will have low vapor pressures and thus have low volatility or are rather non-volatile. Accordingly, it is preferred that the support modifiers are provided in amounts sufficient to: (i) counteract acidic sites present on the surface of the support material; (ii) impart resistance to shape change under hydrogenation temperatures; or (iii) both. Without being bound by theory, imparting resistance to shape change refers to imparting resistance, for example, to sintering, grain growth, grain boundary migration, migration of defects and dislocations, plastic deformation and/or other temperature induced changes in microstructure.

### Catalyst Preparation

The catalyst compositions suitable for use with the present invention preferably are formed through metal impregnation of a support, optionally a modified support. Such impregnation techniques are described, for example, in U.S. Pat. Nos. 7,608,744 and 7,863,489 and U.S. Pub. No. 2010/0197485 referred to above, the entireties of which are incorporated herein by reference, and are further described below. Such processes involve the impregnation of one or more metal precursor solutions onto a catalysts support, typically followed by drying and/or calcination to for the final catalyst.

The catalyst preparation processes of the invention involve the use of one or more acids in the one or more metal precursor solutions, preferably where at least one of the metal precursors is a tin precursor. Some tin precursors, in particular organometallic tin precursors such as tin oxalate, are particularly difficult to solubilize in aqueous systems. It has surprisingly and unexpectedly been discovered that the use of an acid in the tin precursor solution facilitates dissolution.

Thus, in one embodiment, the invention is to a process for preparing a catalyst composition, in which the process includes a step of contacting a catalyst support with a precursor solution comprising a tin precursor, a Group VIII metal precursor, and water at a pH less than 3, e.g., less than 2.5 or less than 2, to form an impregnated support, and drying and/or calcining the impregnated support to form a catalyst comprising tin and a Group VIII metal, e.g., platinum or palladium, on the support.

In another embodiment, the process comprises the step of providing a first solution comprising a tin precursor and an acid selected from nitric acid, hydrochloric acid, sulfuric acid, acetic acid, citric acid, formic acid, lactic acid, malic acid, orthophosphoric acid, salicylic acid, succinic acid, tartaric acid and trichloroacetic acid. A second metal precursor, preferably a Group VIII metal precursor such as platinum or palladium, or a solution thereof is combined with the first solution to form a mixed metal precursor solution. The second metal precursor preferably comprises a second metal oxalate, acetate, halide or nitrate. The process further comprises a step of impregnating a support with the mixed metal precursor solution to form an impregnated support, and drying and/or calcining the impregnated support to form a catalyst comprising tin and the second metal on the support.

The catalyst compositions of the invention preferably are formed through metal impregnation of the support or the modified support, whichever the case may be. Before the metals are impregnated, it typically is desired, for modified supports, to form the modified catalyst support through a step of impregnating the support material with the support modifier. A precursor to the support modifier, such as an acetate or a nitrate, may be used.

In one aspect of the invention, an optional support modifier, e.g., CaSiO₃, is added to the support material, e.g., SiO₂. For example, an aqueous suspension of the support modifier may be formed by adding the solid support modifier to deionized water, followed by the addition of colloidal support material thereto. The resulting mixture may be stirred and added to additional support material using, for example, incipient wetness techniques in which the support modifier is added to a support material having the same pore volume as the volume of the support modifier solution. Capillary action then draws the support modifier into the pores in the support material. The modified support can then be formed by drying and calcining to drive off water and any volatile components within the support modifier solution and depositing the support modifier on the support material. Drying may occur, for example, at a temperature of from 50° C. to 350° C., e.g., from 100° C. to 250° C. or about 120° C., optionally for a period of from 1 to 24 hours, e.g., from 3 to 15 hours or from 6 to 12 hours.

In one aspect, the support particles are shaped, for example, extruded, pelletized, tabletized, pressed, crushed or sieved to form larger agglomerated support particles having the desired size distribution, and the resulting agglomerated support particles may be treated to form the modified support particles. Alternatively, support particles that have already been modified may be shaped into larger agglomerated particles having the desired size distribution, e.g., to form agglomerated particles having an average particle size in the range of from 0.2 to 0.4 cm. Thus, the supports may be extruded, pelletized, tabletized, pressed, crushed or sieved to the desired size distribution before or after the support modification step. Any of the known methods to shape the support materials into desired size distribution can be employed. Calcining of the shaped modified support may occur, for example, at a temperature of from 250° C. to 800° C., e.g., from 300 to 700° C., from 250 to 650° C., or at about 500° C., optionally for a period of from 1 to 12 hours, e.g., from 2 to 10 hours, from 4 to 8 hours or about 6 hours.

In a preferred method of preparing the inventive catalyst, the tin and preferably the second metal are impregnated onto the support, preferably the modified support. A precursor preferably is used in the metal impregnation step, such as a water soluble compound or water dispersible compound/complex that includes the first metal of interest. Depending on the metal precursor employed, the use of a solvent, such as water, glacial acetic acid or an organic solvent, may be preferred.

Tin has a tendency to aggregate in solution, particularly when combined with other metal precursors. Without being bound by theory, it is believed that the use of a catalyst preparation solution having a pH of less than 3, may inhibit the tin from aggregating, which improves dissolution of the tin precursor even when combined with another metal precursor. The use of nitric acid is preferred, although it is contemplated that other acids may be used such as, but not limited to, acids selected from the group consisting of hydrochloric acid, sulfuric acid, acetic acid, citric acid, formic acid, lactic acid, malic acid, orthophosphoric acid, oxalic acid, salicylic acid, succinic acid, tartaric acid, and trichloroacetic acid. This, surprisingly and unexpectedly, allows for remarkable improvements in catalyst performance, namely a significant increase in selectivity to ethanol based on acetic acid used in processes for the production of ethanol, in addition to the high conversions as described above.

In a preferred embodiment, the catalyst impregnation solution is acidified such that it has a pH of less than 3, e.g., less than 2.5 or less than 2. In terms of range, the acidified impregnation solution optionally has a pH from 0.1 to 3, e.g., from 1 to 3, or from 1.5 to 2.5. Thus, the tin and optional second metal are impregnated onto the support in the acidic impregnation solution, which preferably comprises nitric acid.

Without being bound by theory, it is believed that the use of nitric acid (or alternatively a solution having a pH of below 3) in the preparation of the catalyst composition may, inter alia, inhibit or prevent the tin from aggregating, which may allow for better catalyst performance, namely a significant increase in selectivity to ethanol based on acetic acid used in processes for the production of ethanol, in addition to the high conversions as described above. The increased selectivity also desirably results in improved ethanol productivity.

Impregnation as a means of supported catalyst preparation is generally achieved by filling the pores of a support or modified support with a solution of a metal salt (from which the solvent is subsequently evaporated). In particular, impregnation may occur by adding, optionally drop wise or by spraying, either or both the tin precursor and/or the second metal precursor and/or additional metal precursors, preferably in suspension or solution, to the dry modified support. The resulting mixture may then be dried and heated, e.g., optionally under vacuum, in order to remove the solvent. Additional drying and calcining may then be performed, optionally with ramped heating to form the final catalyst composition. Upon heating and/or the application of vacuum, the metal(s) of the metal precursor(s) preferably decompose into their elemental (or oxide) form. In some cases, the completion of removal of the liquid carrier, e.g., water, may not take place until the catalyst is placed into use and calcined, e.g., subjected to the high temperatures encountered during operation. During the calcination step, or at least during the initial phase of use of the catalyst, such compounds are converted into a catalytically active form of the metal or a catalytically active oxide thereof.

In the first step of impregnation, three general processes occur: (1) transport of solute to the pore system of the support bodies; (2) diffusion of solute with the pore system; and (3) uptake of solute by the pore wall. When several precursors are present simultaneously in the impregnating solution, the impregnation is called “co-impregnation.” Thus, impregnation of tin and the second metal into the modified support may occur simultaneously (co-impregnation) or sequentially. In simultaneous impregnation, the tin and second metal precursors are mixed together and added to the modified support together, followed by drying and calcination to form the final catalyst composition. With simultaneous impregnation, it may be desired to employ a dispersion agent, surfactant, or solubilizing agent, e.g., ammonium oxalate, to facilitate the dispersing or solubilizing of the tin and second metal precursors in the event the two precursors are incompatible with the desired solvent, e.g., water. In other embodiments, the precursor solution is substantially free of solubilizing agents such as ammonium oxalate.

In sequential impregnation, the tin precursor may be first added to the support, optionally modified support, followed by drying and calcining, and the resulting material is then impregnated with the second precursor followed by an additional drying and calcining step to form the final catalyst composition. In other sequential impregnation embodiments, the tin is added to the modified support after impregnation of the second metal precursor. Of course, combinations of sequential and simultaneous impregnation may be employed if desired. Moreover, in instances where a high loading is needed, successive impregnations (and heat treatments) may be utilized in accordance with the inventive process.

Suitable metal precursors include, for example, metal oxalates, acetates, halides, nitrates, and amine solubilized metal hydroxides. Exemplary tin precursors include tin oxalate, tetrabutyl tin, tetramethyl tin (TMT) and tin tetracholoride. Exemplary second metal precursors include metal halides, amine solubilized metal hydroxides, metal acetates, metal nitrates and metal oxalates.

### Ethanol Production

As indicated above, the catalysts formed by the novel processes of the invention are preferably employed in the formation of alcohols, such as ethanol, preferably via the hydration of one or more oxygenates, such as acetic acid. One advantage of catalysts of the present invention is the stability or activity of the catalyst for producing ethanol. Accordingly, it can be appreciated that the catalysts of the present invention are fully capable of being used in commercial scale industrial applications for the hydrogenation of acetic acid, particularly in the production of ethanol. In particular, it is possible to achieve such a degree of stability such that catalyst activity will have rate of productivity decline that is less than 6% per 100 hours of catalyst usage, e.g., less than 3% per 100 hours or less than 1.5% per 100 hours. Preferably, the rate of productivity decline is determined once the catalyst has achieved steady-state conditions.

The process of hydrogenating acetic acid to form ethanol according to one embodiment of the present invention may be conducted in a variety of configurations using a fixed bed reactor or a fluidized bed reactor. In many embodiments of the present invention, an “adiabatic” reactor can be used; that is, there is little or no need for internal plumbing through the reaction zone to add or remove heat. Alternatively, a shell and tube reactor provided with a heat transfer medium can be used. In many cases, the reaction zone may be housed in a single vessel or in a series of vessels with heat exchangers therebetween. It is considered significant that acetic acid reduction processes using the catalysts of the present invention may be carried out in adiabatic reactors as this reactor configuration is typically far less capital intensive than tube and shell configurations.

Typically, the catalyst is employed in a fixed bed reactor, e.g., in the shape of an elongated pipe or tube where the reactants, typically in the vapor form, are passed over or through the catalyst. Other reactors, such as fluid or ebullient bed reactors, can be employed, if desired. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably the reaction is carried out in the vapor phase under the following conditions. The reaction temperature may the range from of 125° C. to 350° C., e.g., from 200° C. to 325° C., from 225° C. to about 300° C., or from 250° C. to about 300° C. The pressure may range from 10 KPa to 3000 KPa (about 0.1 to 30 atmospheres), e.g., from 50 KPa to 2300 KPa, or from 100 KPa to 1500 KPa. The reactants may be fed to the reactor at a gas hourly space velocities (GHSV) of greater than 500 hr⁻¹, e.g., greater than 1000 hr⁻¹, greater than 2500 hr⁻¹ and even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 5:1. Most preferably, the molar ratio of hydrogen to acetic acid is greater than 4:1, e.g., greater than 5:1 or greater than 10:1.

Contact or residence time can also vary widely, depending upon such variables as amount of acetic acid, catalyst, reactor, temperature and pressure. Typical contact times range from a fraction of a second to more than several hours when a catalyst system other than a fixed bed is used, with preferred contact times, at least for vapor phase reactions, from 0.1 to 100 seconds, e.g., from 0.3 to 80 seconds or from 0.4 to 30 seconds.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide, methane, and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is transferred to the vapor state by passing hydrogen, recycle gas, another suitable gas, or mixtures thereof through the acetic acid at a temperature below the boiling point of acetic acid, thereby humidifying the carrier gas with acetic acid vapors, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syn gas”) that is derived from any available carbon source. U.S. Pat. No. 6,232,352, the disclosure of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syn gas is diverted from the methanol synthesis loop and supplied to a separator unit to recover CO and hydrogen, which are then used to produce acetic acid. In a similar manner, hydrogen for the hydrogenation step may be supplied from syn gas.

In some embodiments, some or all of the raw materials for the above-described acetic acid hydrogenation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof.

Methanol carbonylation processes suitable for production of acetic acid are described in U.S. Pat. Nos. 7,208,624, 7,115,772, 7,005,541, 6,657,078, 6,627,770, 6,143,930, 5,599,976, 5,144,068, 5,026,908, 5,001,259, and 4,994,608, the entire disclosures of which are incorporated herein by reference. Optionally, the production of ethanol may be integrated with such methanol carbonylation processes.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syn gas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethoxylates is at least 60%, e.g., at least 70%, or at least 80%. As used herein, the term “ethoxylates” refers specifically to the compounds ethanol, acetaldehyde, and ethyl acetate. Preferably, the selectivity to ethanol is at least 60%, e.g., at least 70%, at least 75%, at least 80%, at least 85%, at least 90%, or at least 95%.

Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. Catalysts formed under acidic conditions according to the present invention surprisingly and unexpectedly provide high productivities of ethanol. In one embodiment, the catalysts enable the hydrogenation of acetic acid to ethanol at an ethanol productivity of at least at least 200 grams of ethanol per kilogram catalyst per hour, e.g., at least 400 grams of ethanol per kilogram catalyst per hour or at least 600 grams of ethanol per kilogram catalyst per hour. Productivities greater than 700 grams of ethanol per kilogram catalyst per hour have also been demonstrated. In terms of ranges, the productivity preferably is from 200 to 3,000 grams of ethanol per kilogram catalyst per hour, e.g., from 400 to 2,500 grams of ethanol per kilogram catalyst per hour or from 600 to 2,000 grams of ethanol per kilogram catalyst per hour.

In various embodiments of the present invention, the crude ethanol product produced by the hydrogenation process, before any subsequent processing, such as purification and separation, will typically comprise unreacted acetic acid, ethanol and water. As used herein, the term “crude ethanol product” refers to any composition comprising from 5 to 70 wt. % ethanol and from 5 to 35 wt. % water. In some embodiments, the crude ethanol product comprises ethanol in an amount from 5 wt. % to 70 wt. %, e.g., from 10 wt. % to 60 wt. %, or from 15 wt. % to 50 wt. %, based on the total weight of the crude ethanol product. Preferably, the crude ethanol product contains at least 10 wt. % ethanol, at least 15 wt. % ethanol or at least 20 wt. % ethanol. The crude ethanol product typically will further comprise unreacted acetic acid, depending on conversion, for example, in an amount of less than 90 wt. %, e.g., less than 80 wt. % or less than 70 wt. %. In terms of ranges, the unreacted acetic acid is preferably from 0 to 90 wt. %, e.g., from 5 to 80 wt. %, from 15 to 70 wt. %, from 20 to 70 wt. % or from 25 to 65 wt. %. As water is formed in the reaction process, water will be present in the crude ethanol product, for example, in amounts ranging from 5 to 35 wt. %, e.g., from 10 to 30 wt. % or from 10 to 26 wt. %.

Ethyl acetate may also be produced during the hydrogenation of acetic acid, or through side reactions and may be present, for example, in amounts ranging from 0 to 20 wt. %, e.g., from 0 to 15 wt. %, from 1 to 12 wt. % or from 3 to 10 wt. %. In addition, acetaldehyde may be produced through side reactions, and may be present, for example, in the crude ethanol product in amounts ranging from 0 to 10 wt. %, e.g., from 0 to 3 wt. %, from 0.1 to 3 wt. % or from 0.2 to 2 wt. %. Other components, such as, for example, esters, ethers, aldehydes, ketones, alkanes, and carbon dioxide, if detectable, collectively may be present in amounts less than 10 wt. %, e.g., less than 6 wt. % or less than 4 wt. %. In terms of ranges, these other components may be present in an amount from 0.1 to 10 wt. %, e.g., from 0.1 to 6 wt. %, or from 0.1 to 4 wt. %. Exemplary component ranges for the crude ethanol product are provided in Table 1.

TABLE 1
CRUDE ETHANOL PRODUCT COMPOSITIONS

| | Conc. | Conc. | | |
|---|---|---|---|---|
| Component | (wt. %) | (wt. %) | Conc. (wt. %) | Conc. (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 0 to 20 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

Ethanol may be recovered from the crude ethanol product using one or more distillation columns. The final ethanol product produced by the process of the present invention may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 2.

TABLE 2
FINISHED ETHANOL COMPOSITIONS

| Component | Conc. (wt. %) | Conc. (wt. %) | Conc. (wt. %) |
|---|---|---|---|
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit as discussed above. In such embodiments, the ethanol concentration of the ethanol product may be higher than indicated in Table 2, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircrafts. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entireties of which are hereby incorporated herein by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Yin U.S. Pat. No. 3,130,007, the entireties of which are hereby incorporated herein by reference.

### EXAMPLES

The present invention will be better understood in view of the following examples, which should be considered non-limiting and provided by way of illustration only.

Single impregnation and multi-step impregnation of platinum-tin catalysts were synthesized and compared by two different catalyst synthesis methods. Method A (comparison) used metal oxalates without acidifying the impregnation solution, while Method B employed an acidified impregnation solution according to exemplary embodiments of the invention.

The single impregnation platinum-tin catalyst was prepared under Method A by first dissolving ammonium oxalate hydrate in deionized H₂O, Solid tin (II) oxalate was added to the solution and dissolved with stirring. Separately, platinum oxalate solution (10 wt. % Pt) was diluted with deionized H₂O. Next, the platinum oxalate solution was added to the solution of the tin oxalate, and the resulting mixture was stirred for 5 minutes at room temperature. In the single impregnation process, the platinum precursor was provided in an amount sufficient to form catalysts having 1.0 wt. % platinum, and 0.5 wt. % platinum. The solution was then used to impregnate a SiO₂—CaSiO₃ catalyst support comprising 6 wt. % CaSiO₃, following by drying under vacuum and at 120° C. under flowing air. The dried composition was calcined at 350° C. for 6 hours under flowing air.

The multi-step impregnation platinum-tin catalyst was prepared under Method A using a two-step impregnation for the addition of the active metals. First, ammonium oxalate hydrate was dissolved in deionized H₂O. Next, solid tin (II) oxalate was added to the solution and dissolved with stirring. Separately, platinum oxalate solution (10 wt. % Pt) was diluted with deionized H₂O. The platinum oxalate solution was added to the solution of the tin oxalate, and the resulting mixture was stirred for 5 minutes at room temperature. The platinum precursor was provided in an amount effective to form a catalyst having a platinum loading of 1.0 wt. %. The solution was then used to impregnate the catalyst support, following by drying under vacuum, and at 120° C. under flowing air. A second impregnation solution was separately prepared in a similar manner, and was stirred for 5 minutes at room temperature. The second solution was used to impregnate, in a second impregnation step, the dried single-impregnated catalyst support, following by drying under vacuum and at 120° C. under flowing air. The final, dried, twice impregnated composition was then calcined at 350° C. for 6 hours under flowing air.

In the Method B single step impregnation process, a precursor solution was prepared by dissolving tin (II) oxalate in a 8 M nitric acid solution at a pH less than 3. Separately, platinum oxalate solution (10 wt. % Pt) was diluted with deionized H₂O. The platinum oxalate solution was added to the tin oxalate solution, and the resulting mixture was stirred for 5 minutes at room temperature. In the single impregnation process, the platinum precursor was provided in an amount sufficient to form catalysts having 1.0 wt. % platinum, and 0.5 wt. % platinum. The solution was then used to impregnate a SiO₂—CaSiO₃ catalyst support comprising 6 wt. % CaSiO₃, following by drying under vacuum and at 120° C. under flowing air. The dried composition was calcined at 350° C. for 6 hours under flowing air.

In the multi-step impregnation process of Method B, a precursor solution was prepared by dissolving tin (II) oxalate in a 8 M nitric acid solution at a pH less than 3. Separately, platinum oxalate solution (10 wt. % Pt) was diluted with deionized H₂O. Next, the platinum oxalate solution was added to the tin oxalate solution, and the resulting mixture was stirred for 5 minutes at room temperature. The platinum precursor was provided in an amount effective to form a catalyst having a platinum loading of 1.0 wt. %. The solution was then used to impregnate a SiO₂—CaSiO₃ catalyst support comprising 6 wt. % CaSiO₃, following by drying under vacuum and at 120° C. under flowing air. A second impregnation solution was separately prepared in a similar manner, and was stirred for 5 minutes at room temperature. The second solution was used to impregnate, in a second impregnation step, the dried single-impregnated catalyst support, following by drying under vacuum and at 120° C. under flowing air. The final, dried, twice impregnated composition was then calcined at 350° C. for 6 hours under flowing air.

The conversion and selectivity of the single- and dual-impregnated catalysts formed by Methods A and B were then tested in a microreactor. Table 3 shows the catalytic activities for the catalyst formed under Methods A and B.

TABLE 3
CATALYTIC ACTIVITY

| | Method A | | Method B | |
|---|---|---|---|---|
| | Acetic Acid | Ethanol | Acetic Acid | |
| | Conversion | Selectivity | Conversion | Ethanol |
| Impregnation | (%) | (%) | (%) | Selectivity (%) |
| Single | 57 | 81 | 85 | 82 |
| (1.0 wt. % Pt) | | | | |
| Single | 79 | 45 | 73 | 84 |
| (0.5 wt. % Pt) | | | | |
| Multiple | 71 | 80 | 94 | 71 |

As shown in Table 3, acetic acid conversion surprisingly and unexpectedly improved from 57% for single impregnation at 1 wt. % platinum to 85%, and resulted in a minor increase in ethanol selectivity. For single impregnation at 0.5 wt. % platinum, acetic acid selectivity surprisingly and unexpectedly improved from 45% to 84% with only a minor drop in conversion. For multiple (dual) impregnation at 1 wt. % platinum, acetic acid conversion surprisingly and unexpectedly increased from 71% to 94% with only a minor drop in ethanol selectivity.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. Furthermore, those of ordinary skill in the art will appreciate that the foregoing description is by way of example only, and is not intended to limit the invention.

We claim:

1. A process for preparing a catalyst, the process comprising:
(a) contacting a catalyst support with a precursor solution comprising a tin precursor, a Group VIII metal precursor, and water at a pH less than 3 to form an impregnated support; and
(b) drying the impregnated support to form a catalyst comprising tin and a Group VIII metal on the support.

2. The process of claim 1, wherein the precursor solution has a pH less than 2.5.

3. The process of claim 1, wherein the precursor solution comprises nitric acid.

4. The process of claim 1, wherein the precursor solution comprises an acid selected from the group consisting of: hydrochloric acid, sulfuric acid, acetic acid, citric acid, formic acid, lactic acid, malic acid, orthophosphoric acid, salicylic acid, succinic acid, tartaric acid, and trichloroacetic acid.

5. The process of claim 1, wherein the catalyst has a productivity for ethanol of at least 700 grams of ethanol per kg catalyst per hour.

6. The process of claim 1, wherein the contacting step is performed by spraying the catalyst support with the precursor solution.

7. The process of claim 1, wherein the contacting step is performed by incipient wetness techniques.

8. The process of claim 1, wherein the contacting step is performed by adding the catalyst support to the precursor solution.

9. The process of claim 1, wherein the catalyst support is selected from the group consisting of silica, alumina, zirconia, magnesia, titania, calcium silicate, aluminosilicates, zeolitic materials, and mixtures thereof.

10. The process of claim 9, wherein the catalyst support comprises a mixture of silica and calcium silicate.

11. The process of claim 1, wherein the catalyst comprises from 0.1 wt. % to 10 wt. % tin.

12. The process of claim 1, wherein the Group VIII metal is platinum or palladium, and the catalyst comprises the Group VIII metal in an amount from 0.1 to 10 wt. %.

13. The process of claim 1, wherein the precursor solution further comprises a third metal precursor selected from the group consisting of palladium, rhodium, ruthenium, iridium, chromium, cobalt, copper, molybdenum, tungsten, vanadium and zinc.

14. The process of claim 1, further comprising the step of calcining the catalyst to form a calcined catalyst.

15. The process of claim 14, wherein the calcining step is conducted at a temperature of between 250° C. and 650° C.

16. A catalyst formed by the process of claim 1.

17. A process for preparing a catalyst composition, the process comprising:
(a) providing a first solution comprising a tin precursor and an acid selected from nitric acid, hydrochloric acid, sulfuric acid, acetic acid, citric acid, formic acid, lactic acid, malic acid, orthophosphoric acid, salicylic acid, succinic acid, tartaric acid and trichloroacetic acid;
(b) combining a second metal precursor or a solution thereof with the first solution to form a mixed metal precursor solution, wherein the second metal precursor comprises a second metal oxalate, acetate, halide or nitrate;
(c) impregnating a support with the mixed metal precursor solution to form an impregnated support; and
(d) drying the impregnated support to form a catalyst comprising tin and the second metal on the support.

18. The process of claim 17, wherein the precursor solution has a pH less than 3.0.

19. The process of claim 17, wherein the precursor solution comprises nitric acid.

20. The process of claim 17, wherein the catalyst has a productivity for ethanol of at least 700 grams of ethanol per kg catalyst per hour.

21. The process of claim 17, wherein the contacting step is performed by spraying the catalyst support with a solution of the precursor solution.

22. The process of claim 17, wherein the contacting step is performed by adding the catalyst support to the precursor solution.

23. The process of claim 17, wherein the support is selected from the group consisting of silica, alumina, zirconia, magnesia, titania, calcium silicate, aluminosilicates, zeolitic materials, and mixtures thereof.

24. The process of claim 17, wherein the support comprises a mixture of silica and calcium silicate.

25. The process of claim 17, wherein the catalyst comprises from 0.1 to 10 wt. % tin.

26. The process of claim 17, wherein the catalyst comprises from 0.1 to 10 wt. % palladium or platinum.

27. The process of claim 17, wherein the precursor solution further comprises a third metal precursor selected from the group consisting of palladium, rhodium, ruthenium, iridium, chromium, cobalt, copper, molybdenum, tungsten, vanadium and zinc.

28. The process of claim 17, further comprising the step of calcining the catalyst to form a calcined catalyst.

29. The process of claim 28, wherein the calcining step is conducted at a temperature of between 250° C. and 650° C.

30. A catalyst formed by the process of claim 17.

31. A process for producing ethanol comprising the steps of:
(a) contacting a catalyst support with a catalyst precursor mixture comprising a tin precursor and a second metal precursor in an acidic impregnation solution such that pores of the catalyst support are filled with the catalyst precursor mixture; and
(b) drying the catalyst precursor mixture to form a catalyst composition comprising tin and the second metal; and
(c) hydrogenating a feed stream comprising acetic acid and hydrogen over the catalyst composition to produce a crude ethanol product stream, wherein the impregnation solution comprises nitric acid.

32. The process of claim 31, wherein the selectivity to ethanol based on acetic acid consumed is at least 60%.

33. The process of claim 31, wherein the selectivity to ethanol based on acetic acid consumed is at least 80%.

34. The process of claim 31, wherein the catalyst precursor mixture has a pH less than 3.0.

35. The process of claim 31, wherein the catalyst precursor mixture comprises nitric acid.

36. A process for producing ethanol comprising the steps of:
(a) providing a catalyst composition comprising tin and a second metal, the catalyst composition being formed by contacting a catalyst support with a catalyst precursor mixture comprising a tin precursor and a second metal precursor in an acidic impregnation solution such that pores of the catalyst support are filled with the catalyst precursor mixture, and drying the catalyst precursor mixture to form the catalyst composition; and
(b) hydrogenating a feed stream comprising acetic acid and hydrogen over the catalyst composition to produce a crude ethanol product stream.

37. The process of claim 36, wherein the catalyst precursor mixture has a pH less than 3.0.

38. The process of claim 36, wherein the catalyst precursor mixture comprises nitric acid.
